(19) United States
(12) Patent Application Publication
(10) Pub. No.: US 2013/0177975 A1
(43) Pub. Date: Jul. 11, 2013

(54) DEVICE FOR CONTROLLING THE TEMPERATURE OF A DIRECT-ILLUMINATION SOLAR PHOTOBIOREACTOR

(71) Applicants: Vincent Goetz, Pollestres (FR); Jeremy Pruvost, Saint-Brevin Les Pins (FR); Jack Legrand, Saint Nazaire (FR); Gael Plantard, Narbonne (FR)

(72) Inventors: Vincent Goetz, Pollestres (FR); Jeremy Pruvost, Saint-Brevin Les Pins (FR); Jack Legrand, Saint Nazaire (FR); Gael Plantard, Narbonne (FR)

(73) Assignees: Universite de Nantes, Saint Nazaire (FR); Centre National de la Recherche Scientifique (CNRS, Paris (FR)

(21) Appl. No.: 13/822,632

(22) PCT Filed: Sep. 13, 2011
(86) PCT No.: PCT/EP2011/065874

§ 371 Date: Mar. 12, 2013

(30) Foreign Application Priority Data
Sep. 13, 2010 (FR) 1057285

(51) Int. Cl. C12M 1/00

(52) U.S. Cl. CPC C12M 21/02
USPC 435/292.1

(57) ABSTRACT

The invention relates to a photoreactor (1) comprising a contained reaction chamber (15), wherein the chamber (15) is separated from the exterior by a light-capturing wall (11) and another wall (12), the capturing wall and the other wall being parallel to one another; characterized in that the photoreactor (1) additionally comprises a thermal valve (13) placed against the other wall (12) for passively controlling the increase in heat inside the chamber (15) due to the radiation passing through the capturing wall (11) in order to maintain the temperature in at least one part of the chamber (15) under a threshold temperature (Ts), the thermal valve (13) being made of a phase-change material.

### FIELD OF THE INVENTION

The invention relates to the field of photoreactors. More particularly, the invention relates to the field of photoreactors comprising a contained reaction chamber. It applies in particular to flow photobioreactors for the flow of a liquid in closed loop. It is not however limited to this precise application and also encompasses for example immobilized cell reactors, without circulation loop, and open loop reactors.

### TECHNOLOGICAL BACKGROUND

The production of biomass by culture of photosynthetic microorganisms via the direct use of solar energy falls perfectly within the framework of sustainable development. This production is possible thanks to direct sunlight capturing photobioreactors, in which sunlight is captured by a capturing surface and returned to the microorganisms, which consume part of this solar radiation for their photosynthesis. The use of a closed photobioreactor comprising a contained reaction chamber, as opposed to open basin reactors, makes it possible to optimise production thanks to the possibility of controlling the growth conditions of the microorganisms (particularly input of various gases and nutrients).

However, closed direct sunlight capturing photobioreactors are capable of undergoing excessive heating up of the microorganism culture. This is all the more true since the culture volume per capture surface is low in this type of installation (for example, but non-limiting, of the order of several litres per square metre of illuminated surface). Also, the microorganisms undergo variations in the amount of sunshine (nycthemeral and annual cycle). Yet, control of the temperature constitutes a key point for the correct operation of photobioreactors. This temperature needs to be controlled so that it lies ideally around the growth optimum of the cultivated microorganism (usually situated between 25° C. and 40° C.). If the temperature is too high, this can cause the death of the microorganisms.

Solutions exist that concern mainly the problem of overheating of the closed direct sunlight capturing photobioreactor.

One solution consists in regularly spraying the photobioreactor with water. Another solution consists in immersing, at least partially, the photobioreactor in a water basin.

Both of these solutions share the drawback of consuming a lot of water due to the phenomenon of evaporation and require the construction of basins.

Moreover, spraying water on the photobioreactor causes fouling of the light capturing surfaces by deposition of mineral salts on said surfaces. The luminous flux reaching the culture is thus reduced.

Basin immersion causes problems of reflection-absorption of part of the luminous flux, also reducing the capturing efficiency of the photobioreactor.

Document FR-A-2914315 thus describes a plant for photosynthesis of algae microorganisms comprising a device for spraying water on the pipes, for reducing the temperature of the culture liquid.

Document US-2008/0160591 describes a photobioreactor placed in a water basin for the purposes of thermal regulation.

Document WO-2008/008262 describes a photobioreactor comprising a heat transfer assembly based on water spray means or a fountain.

Other solutions involve the input of electrical energy for active cooling and/or heating of the culture.

Yet, in the case of a microorganisms culture for the production of energy, it is vital and essential to minimise all energy costs linked to the production of micro-organisms.

Examples of such solutions will be found in documents WO-2007/129327 (which discloses a heat regulation system implementing an external exchanger), US-2008/0220515 (which provides a heat exchanger with an external regulation device), FR-2823761 (which proposes a photobioreactor comprising a double external translucent envelope enabling the circulation of thermoregulation fluids), EP-1928994 (which advocates a heat regulation implementing heat barriers associated with tubes of the reactor, based on sand, SiO₂, glass, plastic or translucent ceramic), EP-0647707 (which describes a photobioreactor comprising heat conducting walls adapted to be heated or cooled directly) and U.S. Pat. No. 4,233,958 (which describes a dome resting on a base plate forming a heat accumulator).

### SUMMARY OF THE INVENTION

One of the objectives of the invention is to overcome at least one of the drawbacks of the prior art described above.

To this aim, the invention provides a photoreactor comprising a contained reaction chamber, wherein the chamber is separated from the exterior by a light-capturing wall and another wall, the capturing wall and the other wall being parallel to each other;

characterised in that the photoreactor additionally comprises a thermal valve placed against the other wall for passively controlling the increase in heat inside the chamber due to the radiation passing through the capturing wall for maintaining the temperature in at least one part of the chamber under a threshold temperature, the thermal valve being made of a phase-change material.

One advantage of this passive heat regulation photoreactor resides in the fact that it does not require either input of energy or water to enable a passive regulation of the temperature within the microorganisms culture.

Other optional and non-limiting features are as follows:
the reactor is a photobioreactor;
the material is a paraffin; which preferably has a phase-change temperature range between 25° C. and 40° C.

### DESCRIPTION OF DRAWINGS

Other objectives, features and advantages of the present invention will become clear on reading the detailed description that follows, with reference to the illustrating and non-limiting drawings, among which:

FIG. 1 is a first example of embodiment of the photobioreactor according to the invention;

FIG. 2 is a second example of embodiment of the photobioreactor according to the invention;

FIG. 3 is a third example of embodiment of the photobioreactor according to the invention;

FIG. 4 is a fourth example of embodiment of the photobioreactor according to the invention; and

FIGS. 5a to 5d show four diagrams illustrating the curves of daily temperature within the photobioreactor of FIG. 1 as a function of the mass of phase-change material used for the thermal valve;

FIGS. 6a to 6c show three diagrams illustrating the curves of daily temperature within the photobioreactor of FIG. 1 as a function of the rear face exchange conditions;

FIG. 7a shows a curve illustrating the evolution of the irradiation power density during an average day in the month of July in Nantes taken as standard day for the diagrams of FIGS. 5a to 5d and 6a to 6c;

FIG. 7b shows a curve illustrating the evolution of the temperature during an average day in the month of July in Nantes taken as standard day for the diagrams of FIGS. 5a to 5d and 6a to 6c;

FIG. 8 schematically represents a vertical sectional view of a variant of embodiment of a photobioreactor according to the present invention, comprising a heat exchanger; and

FIG. 9 represents an example of transmission curve of an infrared radiation filtering glass, capable of being used within the scope of the present invention.

### DETAILED DESCRIPTION

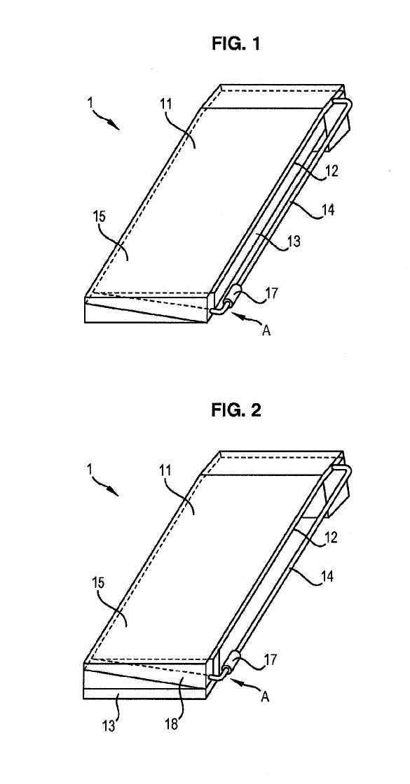

With reference to FIGS. 1 to 4, an example of photobioreactor according to the invention is described hereafter. The photobioreactor 1 enables the culture of one or more types/species of microorganism. The term “microorganisms” will be used hereafter in the plural but also encompasses the singular.

The photobioreactor 1 comprises a contained reaction chamber 15, here a flow chamber for the flow of a liquid in closed loop.

The reaction chamber 15 is comprised between two walls:
a light-capturing wall 11 separating it from the exterior, through which solar radiation passes; and
another wall 12 that may be parallel to the capturing wall 11.

The distance between the capturing wall 11 and the other wall 12 is chosen so as to enable a satisfactory flow in the reaction chamber 15, between said two walls 11 and 12.

Closed loop flow is ensured by a liquid lifting mechanism 14, which can be the subject of numerous embodiments well known to those skilled in the art. For example, the lifting mechanism 14 comprises a fluid lift ramp, one end of which is situated downstream of the flow of the culture, in the lower part of the chamber 15, and the other end is situated upstream, in the upper part of the chamber 15. The lifting mechanism 14 also comprises a pump for making the liquid flow towards the upstream of the reaction chamber 15. The pump causes a flow along the lifting ramp in a direction opposite to the flow of the culture. Such a reactor is described in French patent application n^{o} FR0956870.

The photobioreactor 1 additionally comprises, according to the invention, a thermal valve 13 to maintain, in a passive manner, the temperature under a threshold temperature Ts in at least one part of the reaction chamber 15. The thermal valve 13 may be laid against the other wall 12, positioning it either inside the reaction chamber 15, or outside.

The threshold temperature Ts is determined by the microorganisms present in the culture. Thus, the threshold temperature Ts is chosen so as to be under the maximum temperature that the whole of the cultivated microorganisms can withstand. The threshold temperature Ts may be above the maximum temperature that an undesired microorganism within the culture can withstand.

Within the scope of the present invention, the thermal valve 13 is made from an organic or inorganic phase-change material, the phase change temperature of which is adapted to the desired threshold temperature Ts.

The material making up the thermal valve 13 may be formed for example of paraffin.

By way of non-limiting example and in the case of a threshold temperature Ts of 30° C., a material particularly well suited and commercially available is constituted of paraffin RT31 (Rubitherm) which has a melting range from 27 to 31° C. for a melting enthalpy of 170 kJ/kg.

The efficiency of the heat transfer between the culture and the other wall 12 depends on the flow conditions. That between the other wall 12 and the thermal valve 13 depends on an exchange coefficient between the material of the other wall 12 and that of the thermal valve 13 and the heat conductivity of the thermal valve 13.

Within the context of a thermal valve 13 made of phase-change material, the efficiency of the heat transfer between the thermal valve 13 and the other wall 12 is improved if the phase-change material is within a graphite matrix.

Throughout the phase change duration, the temperature of the phase-change material is substantially constant. In other words, if heat has to be applied to reach the phase change temperature, which is preferably comprised between 25° C. and 40° C., the temperature of the material, which does not yet undergo a change of phase, progressively increases until it reaches the phase change temperature. At this temperature, the phase-change material passes from a first state to a second state. As long as material in the first state still remains, the temperature remains at the phase change temperature. The rise in the temperature of the material will only begin once the material is entirely in the second state.

As for the composition of the phase-change material forming the thermal valve 13, in the temperature range 30° C., the following products may be cited:
alkanes or paraffins: n-octadecane, nonadecane, products commercialised under the denomination RT42, RT31 or RT27 (mixtures of paraffin, Rubitherm products);
organic materials other than paraffin: capric acid (CH₃(CH₂)₈COOH), 1-dodecanol (CH₃(CH₂)₁₁OH), octadecyl thioglycolate, methyl palmitate, methyl stearate, ethyl stearate (and mixture of these latter three constituents), lactic acid, vinyl stearate;
inorganic materials: calcium chloride hexahydrate (CaCl₂.6H₂O), manganese nitrate hexahydrate (Mn(NO₃)₂.6H₂O), lithium nitrate trihydrate (LiNO₃.3H₂O),sodium sulphate decahydrate (Na₂SO₄.10H₂O);
inorganic eutectics: calcium chloride with magnesium chloride hexahydrate; calcium nitrate tetrahydrate with zinc nitrate hexahydrate; calcium chloride, sodium chloride and potassium chloride with water; sodium sulphate decahydrate with water.

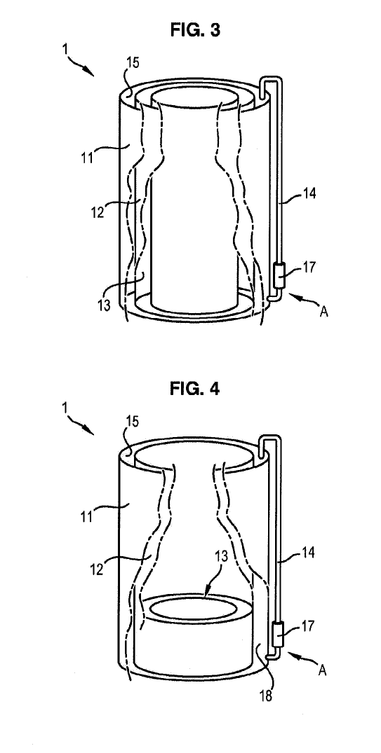

The thermal valve 13 made of phase-change material may cover the whole of the other wall 12. Thus the temperature of the culture is maintained under the threshold temperature Ts throughout the reaction chamber 15 (see FIGS. 1 and 3).

The thermal valve 13 made of phase-change material may only cover a part of the other wall 12, or even be in contact with a part of the photobioreactor 1 which is not in the reaction chamber 15 but in a security chamber 18 downstream of the flow of the liquid that constitutes the microorganisms culture.

In FIGS. 2 and 4, the security chamber 18 corresponds to a compartment situated in the lower part of the photobioreactor 1 and in which the liquid contained in the reaction chamber 15, in the event of interruption of the flow.

The photobioreactor 1 may also comprise a flow regulator 17 to cut off the lifting mechanism 14 and thus the flow loop of the liquid inside the reaction chamber 15 is liable to accumulate when the temperature in the reaction chamber 15 exceeds another threshold temperature Ts′ less than or equal to the threshold temperature Ts. The liquid then accumulates downstream of the flow, potentially in the security chamber 18 if this is provided. Thus, when the temperature in the reaction chamber 15 exceeds the other threshold temperature Ts′, the liquid is contained in a space of the reaction chamber 15 or in the security chamber 18, where the thermal valve 13 (see FIGS. 2 and 4) is located.

The phase-change material is also used as energy storage. Indeed, by heating up, then by changing state, the phase-change material stores up solar energy (the energy due to radiation not consumed by photosynthesis) and releases it when the solar radiation becomes insufficient (for example at the end of the day) ensuring that the optimal growth temperature of the microorganisms is maintained for a longer time.

The photobioreactor 1 may be a flat photobioreactor, as illustrated in FIGS. 1 and 2. In this case, the upper capturing wall 11 and the other lower wall 12 are flat, parallel to each other and sloping in relation to the ground thereby ensuring flow by gravity. The capturing wall 11 is then placed above the other wall 12, this arrangement being imposed by geometry so that sunlight may be directly captured. In the lower part, the bottom of the security chamber 18 moreover comprises a face sloping downwards in the direction of the inlet point of the lifting conduit 14.

The front capturing face 11 is formed typically of a glass window of several mm thickness.

The rear face 12 is formed of a panel of suitable material, for example metal, glass or polymer.

The thermal valve 13 may entirely cover the other wall 12 either above (in which case, the thermal valve 13 is inside the reaction chamber 15), or below (see FIG. 1). The other wall 12 and the thermal valve 13 are in contact with each other to enable heat transfer.

The thermal valve 13 may only cover a security chamber 18 provided in the photobioreactor 1, either above, or below (see FIG. 2). The thermal valve 13 is positioned in contact with a wall of the chamber 18 to ensure heat transfer.

The photobioreactor 1 may also be a cylindrical photobioreactor, as illustrated in FIGS. 3 and 4. In this case, the capturing wall 11 and the other wall 12 have a cylindrical geometry and centred on the same axis, preferably vertical. The capturing wall 11 is outside whereas the other wall 12 is inside, this arrangement being imposed by geometry so that sunlight may be directly captured. The flow of the liquid formed by the microorganism culture takes place from top to bottom by gravity.

The thermal valve 13 may entirely cover the other wall 12 either by the exterior of the cylinder formed by the other wall 12 (in which case, the thermal valve 13 is inside the reaction chamber 15), or by the interior of the cylinder formed by the other wall 12 (in which case, the thermal valve 13 is outside of the reaction chamber 15, see FIG. 3). The other wall 12 and the thermal valve 13 are in contact with each other to enable heat transfer.

The thermal valve 13 may only cover a part of the other wall 12 either by the exterior (see FIG. 4), or by the interior of the cylinder formed by the other wall 12. The thermal valve 13 is positioned in contact with the lower part of the other wall 12 to ensure heat transfer and the security function in this security part. In fact, when the lifting mechanism 14 is cut, the liquid that constitutes the culture flows downwards into the security part 18.

The photobioreactor 1 may further comprise a near infrared and/or ultraviolet filter on or under the light-capturing wall 11. The filter is transparent to wavelengths of the visible domain. Providing a filter is advantageous in that not all of the solar radiation is useful. Indeed, only the part of the solar radiation corresponding to the wavelengths situated in the visible domain is useful to the photosynthetic microorganisms with a maximum efficiency of 15%. A large part of the solar radiation entering into the reactor thus has the consequence of heating up the reaction chamber 15.

The efficiency of heat transfer between the culture and the other wall 12 depends on the flow conditions. The efficiency of heat transfer between the other wall 12 and the thermal valve 13 depends on an exchange coefficient between the material of the other wall 12 and that of the thermal valve 13 and the thermal conductivity of the thermal valve 13.

Within the context of a thermal valve 13 made of phase-change material, the efficiency of heat transfer between the thermal valve 13 and the other wall 12 is improved if the phase-change material is within a graphite matrix.

The remainder of the radiation may be harmful (ultraviolet representing 5% of the total power density of the standard solar spectrum, i.e. around 50 WM⁻²) or may cause overheating of the photobioreactor 1 (near infrared representing 52% of the total power density of the standard solar spectrum, i.e. around 515 Wm⁻²). Moreover, since the capacity of the thermal valve 13 made of phase-change material to maintain the culture chamber 15 under the threshold temperature Ts depends on its mass, providing a filter makes it possible to reduce the necessary mass, since a part of the heating radiation does not enter the reaction chamber 15.

The capturing wall 11 can play the role of filter towards radiation that is not useful. For example, a capturing wall 11 made of conventional glass naturally plays the role of filter for the ultraviolet radiation comprised in the solar spectrum. Technical glasses ensure the function of filter of near infrared radiation (wavelengths comprised between 700 nm and 3000 nm). The percentage of transmission of commercially available glasses intended for the filtration of infrared and near infrared radiation has been represented in appended FIG. 9. Other materials may be used in order to ideally approach a perfect transparency to the wavelengths useful for photosynthesis situated in the visible domain and a total reflection to ultraviolet and especially near infrared wavelengths.

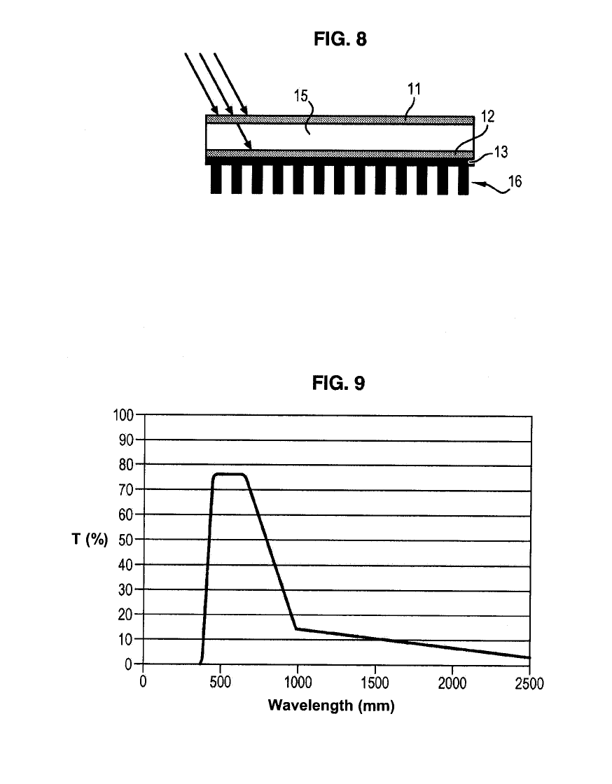

As it is schematically represented in FIG. 8, the photoreactor 1 may also comprise a heat exchanger 16. The heat exchanger 16 may be placed in contact with the thermal valve 13 when it is provided outside of the reaction chamber 15. The heat exchanger 16 may also be placed in contact with the other wall 12, particularly when the thermal valve 13 is provided inside the reaction chamber 15. The heat exchanger 16 makes it possible to relieve the thermal valve 13 by while ensuring a certain cooling thereof.

The heat exchanger 16 may also have another position, as long as it can ensure a heat transfer between the reaction chamber 15 and the exterior through potentially an element of the photobioreactor 1.

The heat exchanger 16 may be a finned radiator.

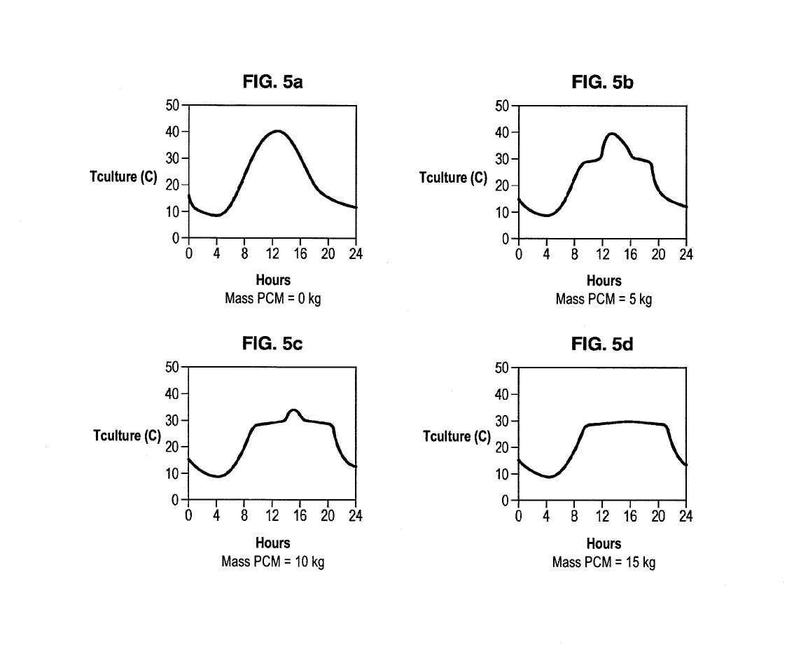

FIGS. 5a to 5d illustrate the results of a theoretical calculation of the heating up of the culture within the reaction chamber 15 during a standard day when the photobioreactor 1 does not comprise a thermal valve 13 (FIG. 5a) or comprises a thermal valve 13 made of phase-change material having a thickness of 1 cm (FIG. 5b), 2 cm (FIGS. 5c) and 3 cm (FIG. 5d).

The calculations leading to FIGS. 5a to 5d have been performed for a photobioreactor 1 comprising a glass filter having a transmission and a reflection of solar radiation of 0.5. The exchange conditions with the surrounding medium, the surfaces 11 and 12 (FIG. 5a) or the surface of the thermal valve 13 (FIG. 5b, 5c, 5d), retained for the calculation, correspond to an exchange coefficient by natural convection of 5 Wm⁻²K⁻¹.

The thermal valve used in the simulation comprises a surface area of 0.7 m², and is made of phase-change material formed of Rubitherm having respectively thicknesses of 1 cm, 2 cm and 3 cm corresponding to masses of 5 kg, 10 kg and 15 kg.

It may be seen in FIG. 5a that when the photobioreactor 1 does not comprise a thermal valve 13 (FIG. 5a), the temperature within the reaction chamber 15 reaches a maximum of 40° C. between 12:00 and 13:00 and remains greater than 30° C. for nearly seven hours.

When the photobioreactor 1 comprises a thermal valve 13 made of phase-change material of 1 cm (FIG. 5b), the temperature within the reaction chamber 15 also reaches a maximum of 40° C. around 13:00 and remains greater than 30° C. for four hours, i.e. three hours less than in the case illustrated in FIG. 5a.

When the thermal valve 13 is 2 cm (FIG. 5c), the temperature within the reaction chamber 15 remains below 35° C. throughout the day and greater than 30° C. for around two hours and twenty minutes, i.e. nearly four hours and forty minutes less than in the case illustrated in FIG. 5a. The temperature peak is shifted to around 14:45.

Finally, when the thermal valve 13 is 3 cm, the temperature within the reaction chamber 15 remains constantly below 30° C. (FIG. 5d).

These comparisons demonstrate the efficiency of the thermal valve 13 made of phase-change material.

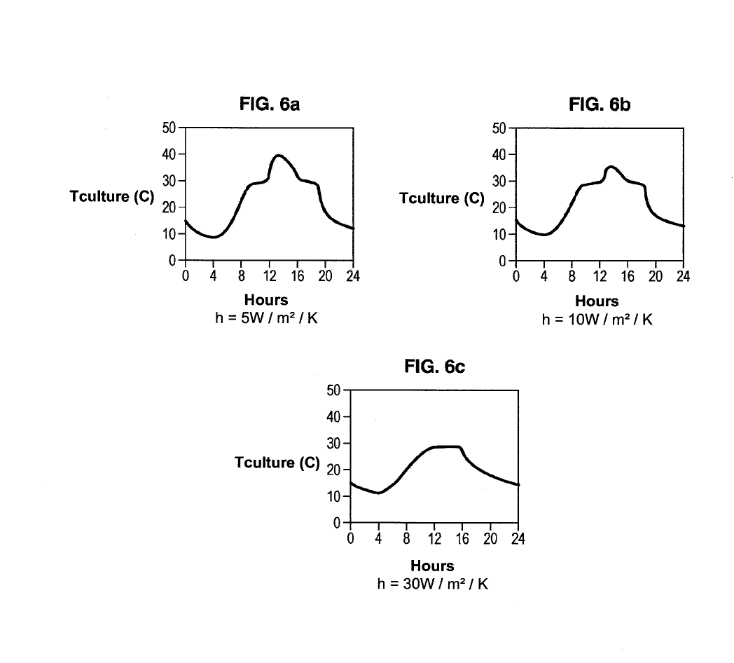

FIGS. 6a to 6c illustrate the heating up of the culture within the reaction chamber 15 during a standard day when the photobioreactor 1 comprises a glass filter, a thermal valve 13 made of phase-change material having a thickness of 1 cm without heat exchanger (FIG. 6a): and with a heat exchanger developing a finned surface (comprising fins) making it possible to increase the initial exchange surface (here 0.7 m²) by a factor 2 (FIG. 6b) or by a factor 6 (FIG. 6c). The calculations presented here for illustrative purposes have been performed in the case of an exchange in conditions of natural convection leading to an exchange coefficient of 5 Wm⁻²K⁻¹.

FIG. 6a corresponds to FIG. 5b. It will thus not be commented on further.

When the rear face heat exchanger makes it possible to double the exchange surface area (FIG. 6b), the temperature within the reaction chamber 15 does not exceed 35° C. throughout the standard day and remains above 30° C. for less than three hours and twenty minutes, approaching the result obtained in the case illustrated by FIG. 5c.

When the rear face heat exchanger develops a surface area six times greater (FIG. 6c), the temperature within the reaction chamber 15 does not exceed 30° C. throughout the standard day.

The result is even improved with respect to the case illustrated by FIG. 5d.

Thus, it may be seen that the use of a rear face heat exchanger improves the results obtained. This makes it possible to reduce the necessary mass of phase-change material.

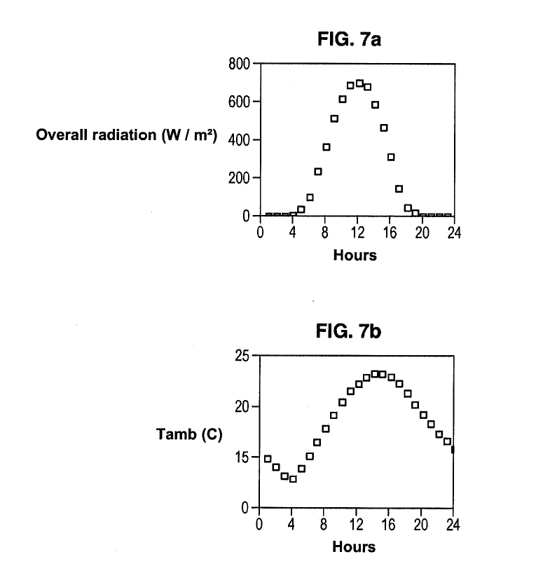

The evolution of the temperature of a standard day as envisaged during simulations ending up with the results of FIGS. 5a to 5d and 6a to 6c is illustrated in FIG. 7b. This evolution corresponds to that of an average day in the month of July in Nantes. The evolution during the day of the flux density corresponding to the solar radiation is illustrated in FIG. 7a.

The above description has been made with reference to a photobioreactor, but it may also be easily adapted to any type of direct sunlight capturing reactor for example a photoreactor operating in the domain of photocatalysis for the treatment of liquids. Also, the geometries may vary and those skilled in the art will know how to adapt the teaching of the present description to these various geometries.

Inputs of gas A, particularly CO₂, and of nutrient take place via dedicated conduits known to those skilled in the art, for example at the conduit 14. In the same way, decanting for the collection of the microorganisms takes place by any appropriate means known to those skilled in the art, for example with the aid of means provided for this purpose on the conduit 14, when the conditions are met.

Those skilled in the art will appreciate on reading the above description that the present invention enables decisive advantages with respect to the regulation of temperature, vis-à-vis the prior art, by implementing a passive regulation system.

Obviously, the present invention is not limited to the particular embodiments that have been described, but extends to all variants compliant with its spirit.

Thus for example it is not necessary to have a pump on the conduit 14 when the liquid is made to move by other means, for example as is known per se, by the differential static pressure resulting from the injection of gas into the reaction chamber 15.

1. A photoreactor comprising a contained reaction chamber, wherein the chamber is separated from the exterior by a light-capturing wall and another wall, the capturing wall and the other wall being parallel to each other;
wherein the photoreactor additionally comprises a thermal valve placed against the other wall for passively controlling the increase in heat inside the chamber due to the radiation passing through the capturing wall for maintaining the temperature in at least one part of the chamber under a threshold temperature, the thermal valve being made of a phase-change material.

2. The photoreactor of claim 1, wherein the reactor is a photobioreactor.

3. The photoreactor of claim 1, wherein the material is a paraffin.

4. The photoreactor of claim 3, wherein the material is a paraffin with a phase-change temperature range close to 30° C.

5. (canceled)

6. The photoreactor claim 1, wherein the capturing wall and the other wall are flat and sloping in relation to the ground thereby ensuring a flow, the capturing wall being placed above the other wall.

7. The photoreactor claim 1, further comprising a filter only allowing useful radiation to pass through.

8. The photoreactor claim 1, further comprising a heat exchanger placed against the thermal valve, if applicable, opposite the other wall and ensuring heat exchange between the thermal valve and the exterior.

9. The photoreactor of claim 8, wherein the heat exchanger is a finned radiator.

10. The photoreactor claim 1, wherein the thermal valve is placed downstream of the flow, and further comprising a flow regulator to cut the flow loop of liquid inside the reaction chamber when the temperature in the reaction chamber exceeds another threshold temperature, the liquid accumulating downstream of the flow.

11. The photoreactor of claim 16, wherein the phase-change material is chosen from the group consisting of n-octadecane, nonadecane, and products made from mixtures of Rubitherm RT42, RT31 and RT27.

12. The photoreactor of claim 1, wherein the phase-change material is chosen from the group consisting of capric acid; 1-dodecanol; octadecyl thioglycolate; methyl palmitate; methyl stearate; ethyl stearate; mixtures of methyl palmilate, methyl stearate and ethyl stearate; lactic acid; and vinyl stearate.

13. The photoreactore of claim 1, wherein the phase-change material is chosen from the group consisting of calcium chloride hexahydrate; manganese nitrate hexahydrate; lithium nitrate trihydrate; and sodium sulphate decahydrate.

14. The photoreactor of claim 1, wherein the phase-change material is an inorganic eutectic.

15. The photoreactor of claim 9, wherein the phase-change material is chosen from the group consisting of mixtures of calcium chloride and magnesium chloride hexahydrate; mixtures of calcium nitrate tetrahydrate and zinc nitrate hexahydrate;
mixtures of calcium chloride, sodium chloride, potassium chloride and water; and mixtures of sodium sulphate decahydrate and water.

16. The photoreactor of claim 1, wherein the phase-change material is alkanes or paraffins.
